(19) United States
(12) Patent Application Publication
(10) Pub. No.: US 2017/0049166 A1
(43) Pub. Date: Feb. 23, 2017

(54) WEARABLE DEVICE AND METHOD FOR SECURING TUBING

(71) Applicant: Danielle L Shorter, Matteson, IL (US)

(72) Inventor: Danielle L Shorter, Matteson, IL (US)

(21) Appl. No.: 14/831,486

(22) Filed: Aug. 20, 2015

(51) Int. Cl. A41D 13/12; A41D 27/20; A61M 25/02

(52) U.S. Cl. CPC A41D 13/1245; A61M 25/02; A41D 27/20; A41D 13/129; A61M 2025/0206; A61M 2209/084

(57) ABSTRACT

The present invention relates to a wearable device particularly adapted for use with an individual connected to a medical device by tubing or a cord, which allows the wearer to move freely, but prevents the tubing from disconnecting from the person or the medical device via a securing attachment that restricts the movement of the wearable device's wearer to a distance shorter than the length of the tubing connecting the wearer to the medical device. The wearable device may also contain containment members to secure excess lengths of tubing. The wearable device can allow access to the wearer's body without removing the wearable device from the wearer.

### BACKGROUND OF THE INVENTION

1. Field of the Invention

The present invention relates to a wearable device particularly adapted for use with a person connected to a medical device by tubing or a cord. The wearable device can enable its wearer to move freely in any direction available to the wearer, but also prevents the tubing from disconnecting from the person or the medical device via a securing attachment that restricts the movement of the wearer to a distance shorter than the length of the tubing connecting the wearer to the medical device. The wearable device can also allow access to the wearer's body without removing the wearable device from the wearer.

2. Background of Related Art

Medical treatment often involves using a medical device that is connected to an individual via a tubing or cord, sometimes for an extended period of time. For example, intravenous (IV) tubing may connect a patient to an IV bag, with the IV tubing transporting fluids and/or medicine from the IV bag to the patient. Similarly, a patient may be connected to a medical monitoring device via a cord, where the cord transmits information between the patient and the monitoring device.

Some medical devices are secured directly to the patient during treatment, such as a catheter attached to a patient's body. This allows a patient to remain ambulatory while keeping the medical device close to the patient throughout the period of treatment. Other medical devices have longer connections, such as tubing or cords, which allow patients to move away from the medical device during treatment.

The tubing or cords which connect a patient to the medical device give the patient a certain degree of movement during treatment. This freedom of movement allows patients to engage in many normal activities during the treatment period, making the medical treatment more bearable for them. This freedom of movement also enables the patient to undergo other medical procedures at the same time.

However, in order to maintain the continued functioning of the medical device, the patient's range of movement is, of necessity, limited to the length of the tubing or cord that connects the patient to the medical device. If the patient strays further than that length away from the medical device, the connecting tubing may be pulled out of the patient, exposing her to injury or infection. Alternatively, the tubing may be pulled from the medical device, possibly damaging the medical device or the tubing itself. At a minimum, the patient's treatment is interrupted, and may have to be repeated from the beginning. Worse, the patient may suffer injury when the device is unexpectedly disconnected or physical harm from the disruption of the medical treatment. While the mobility allowed by the tubing or cords creates many advantages for the patient, it also increases the risk that a person may become disconnected from the medical device or injured during treatment.

Garments have been developed as a strategy to secure patients to medical devices in a way that preserves the patient's mobility while minimizing the risk of disconnecting the patient from the medical device. However, these garments have generally been limited in application to small medical devices or they drastically restrict movement between the patient and the medical device.

During the course of treatment, the patient may require additional examination or treatment, or the medical device may require adjustment or replacement at its site of attachment to the patient. It is a concern that the patient's body remains accessible without disrupting the ongoing treatment or monitoring provided by the medical device and without having to remove the garment, and the protections it confers, while in use.

An additional concern for a person connected to such a medical device is that excess lengths of tubing or cords be kept out of the way so that the patient does not become entangled in the tubing or cords or trip over them. A related concern is that excess lengths of tubing be kept out of the way of other individuals, such as medical personnel, service animals, or visitors, to prevent entanglement, tripping, or disconnection.

An additional consideration includes the ease and speed of disengaging the garment from the medical device when medical treatment has concluded. Patients may wish to leave the medical setting without removing the garment or having to change clothes. Similarly, patients may wish to start treatment without removing their own clothes. A related consideration includes the desire to use familiar articles of clothing during treatment, instead of hospital gowns or institutional garments, preserving the patient's dignity and minimizing the discomfort caused by unfamiliar, possibly ill-fitting institutional garments.

There is a particular need for such wearable devices for children who are connected to a medical device by lengths of tubing or cord. The freedom of movement conferred by the tubing or cords enables children to move, rest, and play during treatment or monitoring, making the treatments less unpleasant for them. Because children may dart away from the medical device, from boredom or inattention, and inadvertently disconnect the tubings or cords from the medical device or themselves, there is a need for a wearable device that protects children from disconnecting from these medical devices while also giving them a degree of mobility during treatment. Such a garment may minimize the unpleasantness children often associate with medical treatments and may improve their compliance with these treatments.

Therefore, a need exists for a wearable device that will provide a person connected to a medical device by tubing or cords with the freedom to move in any direction accessible to the wearer, while preventing the patient from moving far enough away from the medical device to disconnect the tubing, and can allow access to the patient's body without removing the garment, and there is a particular need for such a garment that is suitable for use with children undergoing medical treatment.

### DESCRIPTION OF THE RELATED ART

Traditionally, reusable medical garments have been used to secure IV tubing or catheters to a patient's body. However, this solution has several limitations. These types of garments are limited to medical devices that can be directly secured to the patient. They keep the individuals wearing them in close proximity to the medical device and do not allow the wearer to move even an arm's length away from the medical device when in use. In addition, some of these garments are designed to restrict the wearer's movement in one or more directions.

Reusable medical garments are used to secure catheters, drains, and other medical devices small enough to be supported by the patient's body. They cannot be used or adapted to secure a patient's connection to many larger, heavier, or bulkier medical devices. They cannot be used to secure medical devices that are too large or too heavy for the patient to support. In addition, many of these garments are specifically adapted to prevent the wearer from moving away from the medical device.

Other appliances are used to secure dressings to patients. Like the reusable medical garments, these appliances are also limited to securing small, lightweight medical devices to patients. While they do may substantially impair their wearer's general mobility, they also may not allow the wearers to move any distance away from the dressing.

These alternatives also fail to prevent the disconnection of the tubing from the medical device or the patient. Some garments allow an excess loop of tubing to remain unsecured, in order to minimize the likelihood of the tubing being pulled out of the patient, but do not prevent the patient from moving far enough away from the medical device to dislodge the tubing.

There are a limited number of alternatives for securing medical tubing or cords that connect patients to medical devices, but none of them allow movement in any direction away from the medical device.

Fixed mechanical restraints have been used to prevent a patient from pulling out an IV tubing or detaching a lead from a monitoring device. These alternatives involve mechanical assemblies that maintain a fixed position, or a limited range of positions, between the patient and the medical device. These alternatives rob the patient of the ability to move freely with respect to the medical device dispensing treatment. They limit the directions in which the patient can move generally and severely constrain the patient's ability to move relative to the medical device.

Furthermore, these fixed mechanical restraints do not secure the medical device to the patient. Instead, they secure the medical device to a wheelchair or bed, constraining the patient with a connection to a second medical device, compared to facilitating freedom of movement away from the primary medical device. The patient's confinement to the wheelchair or bed does not allow her to freely walk, stand, or sit, further limiting freedom of movement. For children, these restrictions also limit their ability to play or otherwise amuse themselves during treatment.

Additionally, these fixed mechanical restraints cannot be easily adjusted without disrupting the patient's connection to the primary medical device providing treatment. While in use, these restraints may also interfere with access to the wearers' bodies. These devices also fail to prevent the disconnection of the tubing from the medical device or the patient. They also fail to prevent the disconnection of the tubing by the patient, another individual, or other obstacle.

Other garments have been designed to hold electrical cords away from their wearers, but they do not prevent the wearer from disconnecting the secured electrical cords by simply moving far enough from the outlets to which the electrical cords are connected.

Tethers have been employed to limit a wearer's range of motion from a tether, but have not been employed to secure patients to medical devices. Some of these tethers have features that minimize entanglement of attached cords, but they do not prevent the wearer from disconnecting from anything other than the tether itself. In addition, these tethers do not allow the user to set the maximum distance allowed from the security device; instead, the user is limited to a single preset maximum length that cannot be changed or adjusted.

### BRIEF SUMMARY OF THE INVENTION

It is an object of the embodiments disclosed herein to provide a wearable device for use with an individual connected to a medical device by tubing or a cord, which allows the wearer to move freely in all directions, but prevents the tubing from disconnecting from the wearer or the medical device via a securing attachment that restricts the movement of the wearable device's wearer to a distance shorter than the length of the tubing connecting the wearer to the medical device.

It is also an object of the embodiments disclosed herein to provide a wearable device that can be put on, adjusted, or taken off without disrupting the wearer's connection to the medical device.

It is also an object of the embodiments disclosed herein to provide a wearable device that allows access to the wearer's body without removing the wearable device from the wearer.

### BRIEF DESCRIPTION OF THE DRAWINGS

FIG. 1A shows a front view of a wearable device with both shoulder fasteners closed;

FIG. 1B shows a front view of a wearable device with both shoulder fasteners unfastened;

FIG. 2A shows a rear view of a wearable device with secured containment members; and

FIG. 2B shows a rear view of a wearable device with one containment member opened and another containment member closed, attached to an associated medical device.

### DETAILED DESCRIPTION OF THE INVENTION

While this invention is susceptible of an embodiment in many different forms, there are shown in the drawings and will be described herein in detail specific embodiments thereof with the understanding that the present disclosure is to be considered as an exemplification of the principles of the invention. It is not intended to limit the invention to the specific illustrated embodiments.

It is to be understood that a “medical device” as disclosed herein includes any medical device used for treatment or monitoring a patient where the patient can move a distance of at least an arm's length away from the medical device while in use. Monitoring devices include, but are not limited to, intravenous stands, electroencephalographic (EEG) equipment, electrocardiographic (EKG) devices, dialysis apparatus, respiratory equipment, enteral feeding devices, ventilators, fetal monitors, telemetry transmitters, and oximeters.

It is to be understood that the phrases “tubing” or “cord” as disclosed herein include at least one intravenous tubing, catheter, feeding tubing, surgical drain, and any other type of tubing, wiring, cable, or cord that forms a flexible and detachable connection between a medical device and a patient that the device is treating or monitoring. The terms “tubing” and “cord” are not limited to any number or type of tubing or cords, or for any particular medical function. Unless specified, these terms are interchangeable and include all variations on those terms.

It is to be understood that a “garment” as disclosed herein includes any article of clothing that can be secured to an individual. Such articles of clothing include, but are not limited to, vests, shirts, dresses, gowns, robes, pants, coats, ponchos, onesies, diapers, and bras.

It is to be understood that a “fastening member” or a “fastener” includes any device which fastens one part of a garment to another part of the garment, or to hold the garment in place on the wearer. Such fastening members may include, but are not limited to, hook and loop fasteners, hook and eye fasteners, adhesives, including reusable adhesives, zippers, buttons, snaps, pins, stitching, and clamps. These fastening members are provided merely by way of illustration and are not intended to be limited to the specific examples described.

It is to be understood that a “securing member” includes any device which fastens a garment to a medical device. Such securing members may include, but are not limited to, hook and loop fasteners, hook and eye fasteners, adhesives, including reusable adhesives, zippers, buttons, snaps, pins, stitching, and clamps. These securing members are provided merely by way of illustration and are not intended to be limited to the specific examples described.

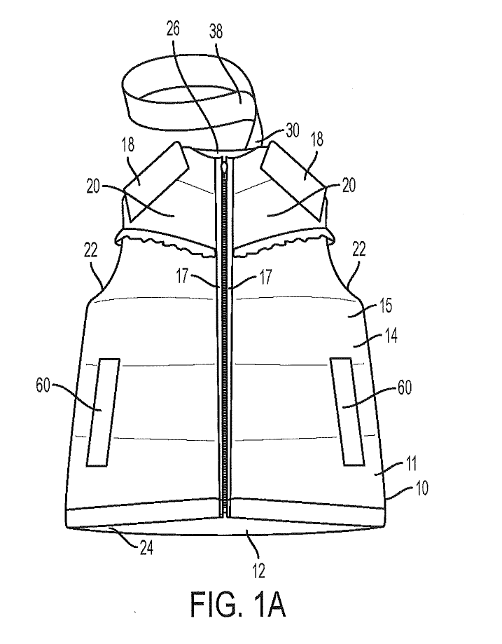

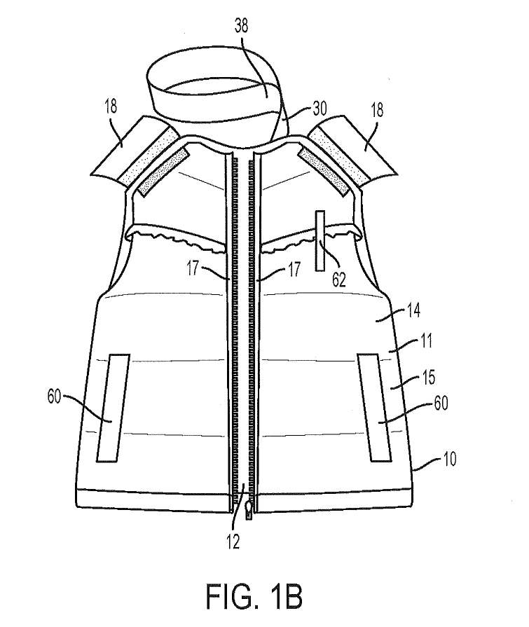

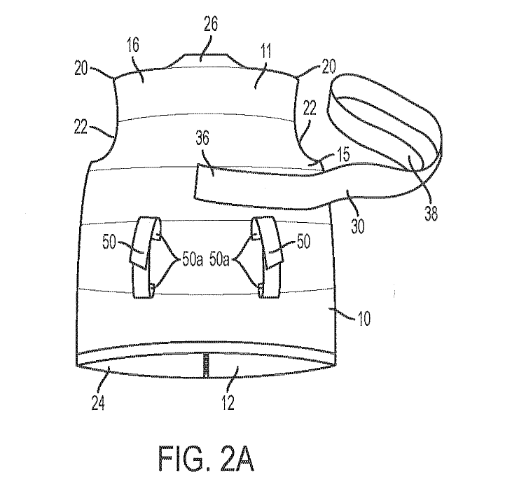

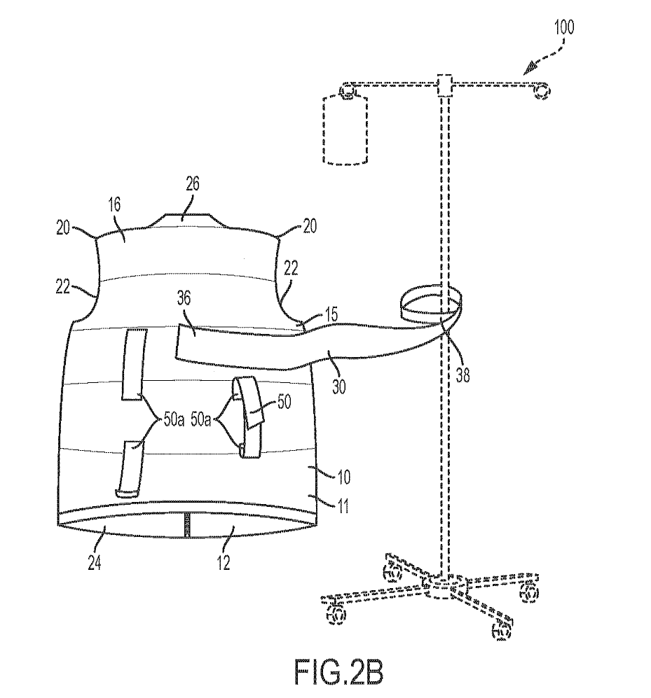

In accordance with disclosed embodiment of the invention, a wearable device is shown in FIGS. 1-2. For example, the wearable device 10 can include a garment 15, such as a vest.

The garment 15 can include an outer surface 11 and an inner surface 12. When the garment 15 is worn, the inner surface 12 can face the wearer. The garment 15 can include a front portion 14, a back portion 16, and two shoulder straps 20 at the top of the garment 15 that join the front portion 14 to the back portion 16. The front portion 14 can include a front fastening member 17 that can be reversibly joined. The shoulder straps 20 can have shoulder fastening members 18 that are reversibly joined. The front fastening member 17 and the shoulder fastening members 18 can be reversibly joined and unfastened as shown in FIGS. 1A-1B, providing access to the wearer's body without removing the wearable device 10.

When the shoulder fastening members 18 are fastened, they define at least one armhole 22 in the garment 15. When the front fastening member 17 is fastened, it can define a bottom opening 24 at the bottom of the garment 15. When the shoulder fastening members 18 are fastened and the front fastening member 17 is fastened, they can define a top opening 26 at the top of the garment 15. When the wearable device 10 is being worn by an individual, a length of tubing may be connected to the wearer through the top opening 26, bottom opening 24, any armhole 22, or any other opening of the garment 15, such as slit 62, and access to the wearer can be accomplished without removing the wearable device 10 by unfastening one or more of the fastening members (for example, 17 or 18), or through slits 62 or other openings.

The wearable device 10 can be reversibly placed onto and removed from the individual without interfering with the treatment or monitoring performed by a medical device 100 and without interfering with the tubing or cords connecting the individual to the medical device. When an individual wears the wearable device 10, she can be connected to a medical device 100 via tubing or cords. However, the wearable device 10 can include a securing member 30 that stops the wearer from moving further away from the medical device 100 than the length of the tubing connecting the wearer to the medical device 100, thus preventing the tubing from being disconnected from the wearer or the medical device 100. In other words, the movement of the wearer can be limited to a distance shorter than the distance equal to the length of the tubing connecting the wearer to the medical device 100, but that distance can be traversed horizontally, vertically, or in any direction away from the medical device 100 that is accessible to the wearer.

The flexible securing member 30 can be made of a flexible material that allows movement in all directions. A first end 36 of the flexible securing member 30 can be attached to the garment 15, for example at the outer surface 11 of the back portion 16. When in use, a second end 38 of the flexible securing member 30 can be removably fastened or connected to the medical device 100 used to treat or monitor the wearer. The second end 38 of the flexible securing member 30 can slidingly attach or connect to adjust the length of the flexible securing member 30 to a length that is shorter than the length of the tubing connecting the individual to the medical device 100. In some embodiments, the slidingly adjustable second end 38 of the flexible securing member 30 can include a hook and loop fastener that can be reversibly fastened and unfastened. The flexible securing member 30 may be attached to the medical device 100 either before or after the wearable device 10 is secured onto the wearer.

One or more containment members 50 can be attached to the wearable device 10, for example at outer surface 11 of the back portion 16 of the garment 15 (FIGS. 2A-2B). The containment members 50 can reversibly secure a portion of tubing to the garment 15, if desired, to prevent the wearer or others from tripping over excess tubing and to prevent inadvertent disconnection of the tubing from either the wearer or the medical device 100. The containment members 50 can be positioned so as not to interfere with the movement of the flexible securing member 30.

The containment members 50 can be reversibly fastened and unfastened. In some embodiments, each containment member 50 can include at least two mating fasteners 50a that can be reversibly joined and unfastened to each other.

In some embodiments, the wearable device 10 can include at least one pocket 60, for example on the outer surface 11 of the front portion 14 of the garment 15.

In some embodiments, the wearable device 10 can include at least one opening, such as a slit 62 that provides an opening between the outer surface 11 and inner surface 12 of the garment 15 to allow access to the wearer's body without removing the wearable device 10.

As shown in FIGS. 1 and 2, some embodiments disclosed herein include a sleeveless vest comprised of cotton, polyester, or other cloth typically used in garments. The vest can close in front by a front fastening member 17, which can include a zipper. After the vest is donned around an upper torso of an individual, the wearer's body can be accessed without removing the vest from its wearer, partially or fully opening the front fastening member 17 or by unfastening at least one shoulder fastening member 18 on the shoulder of the vest, or by use of the slits 62 or other openings.

In some embodiments, the flexible securing member 30 can be reversibly extendable and retractable; that is, the flexible securing member 30 can automatically lengthen as the wearer moves away from the medical device 100 and automatically shorten as the wearer moves toward the medical device 100. In this manner, the risk of disconnection of the flexible securing member 30 may be minimized.

The wearable device 10 as disclosed herein may be used a variety of ways. For example, the flexible securing member 30 may be permanently attached to the wearable device 10, or it may be a detachable element. This would allow an individual to change flexible securing members 30 as needed, for example, for cleaning or repair. A wearer could employ flexible securing members 30 of different lengths, weights, or thicknesses, to accommodate different medical devices, different lengths of tubing, or different ranges of motion for the wearer.

Alternatively, the flexible securing member 30 may be detached, leaving the garment 15 as an ordinary piece of apparel, so that the wearer need not change clothes before or after medical treatment. Similarly, the containment members 50 may be detachable, so that the garment 15 may be worn as an ordinary article of clothing when not in use as a wearable device 10.

In some embodiments, the wearable device 10 may have zero, one, or multiple containment members 50. While FIGS. 2A and 2B show a pair of the containment members 50 on the exterior of the back portion 16 of the garment 15, containment members 50 could be placed on the front, side, or back of the garment 15, so long as such containment members 50 do not interfere with the operation of the flexible securing member 30. In some embodiments of the invention, the containment members 50 include at least two mating fasteners 50a that can be reversibly fastened and unfastened to each other, for example, by hook and loop fasteners. In some embodiments, the containment members 50 may be made of unitary pieces.

Some embodiments disclosed herein can include pockets 60 on the exterior of the front portion 14. Some embodiments of the wearable device 10 can include one or more pockets 60, or none at all. Such pockets could be placed on the interior 12 or exterior 11 of the garment 15 on the front, side, or back, or any other place pockets are generally placed on garments. Similarly, any other common additions to a garment, such as a hood, tabs, drawstrings, or embellishments, may be added.

Although the garment disclosed herein has been described as a sleeveless vest with a zipper, it is to be understood that embodiments disclosed herein are not so limited and can encompass all garments that can be adapted to any article of clothing that can be secured to an individual. Such articles of clothing include, but are not limited to, vests, shirts, dresses, gowns, robes, pants, coats, ponchos, onesies, diapers, and bras.

It is an object of the embodiments disclosed herein to provide a garment for use to prevent disconnection of a patient from a medical or monitoring device. In some embodiments, the use of the garment can prevent the disconnection of an IV tube from a patient or an IV stand. The person of ordinary skill will appreciate the applicability to other medical devices delivering medical treatment and to monitoring devices that may not deliver treatment, but simply monitor some aspect or activity of the wearer.

Some embodiments disclosed herein can be used by a patient in a medical setting. However, similar garments could be used in other settings, for example, in the home. Alternatively, similar garments could be used on animals in medical or monitoring situations.

It is another object of the embodiments disclosed herein to provide a reusable garment that can be easily cleaned, laundered, or sterilized. From the foregoing, it will be observed that numerous variations and modifications may be effected without departing from the spirit and scope of the invention. It is to be understood that no limitation with respect to the specific system or method described herein is intended or should be inferred. It is, of course, intended to cover all such modifications as fall within the spirit and scope of the invention.

What is claimed is:

1. A wearable device adapted to be worn by an individual connected to a medical device via a tubing comprising:
a garment having at least one reversibly joinable fastening member; and
a flexible securing member comprising:
a first end attached to the garment, and
a slidingly adjustable second end capable of being removably connected to the medical device;
wherein the flexible securing member is adjustable to a length that is shorter than a length of the tubing connecting the individual to the medical device; and
wherein the reversibly joinable fastening member provides access to an individual's body without removing the garment.

2. A wearable device as in claim 1 further comprising at least one containment member attached to an outer surface of the garment,
wherein the containment member can reversibly secure a portion of the tubing to the garment and
wherein the containment member is positioned so as not to interfere with a movement of the flexible securing member.

3. A wearable device as in claim 2 wherein the containment member is attached to a back portion of the garment.

4. A wearable device as in claim 1 wherein the flexible securing member may stop the individual before reaching a distance away from the medical device equal to the length of the tubing connecting the individual to the medical device.

5. A wearable device as in claim 1 wherein the wearable device may be reversibly placed onto and removed from the individual without interfering with a treatment performed by the medical device and without interfering with the tubing connecting the individual to the medical device.

6. A wearable device adapted to be worn by an individual connected to a medical device via a tubing comprising:
a garment that encloses at least an upper torso of the individual, the garment having:
at least one top opening at the top of the garment,
at least one bottom opening at the bottom of the garment, and
at least one shoulder strap with a reversibly joinable fastening member; and
a flexible securing member comprising:
a first end attached to the garment, and
a slidingly adjustable second end capable of being removably connected to the medical device;
wherein the flexible securing member is adjustable to a length that is shorter than the length of the tubing connecting the individual to the medical device; and
wherein the reversibly joinable fastening member provides access to an individual's body without removing the garment.

7. A wearable device as in claim 6 further comprising at least one containment member attached to an outer surface of the garment,
wherein the containment member may reversibly secure a portion of the tubing to the garment, and
wherein the containment member is positioned so as not to interfere with a movement of the flexible securing member.

8. A wearable device as in claim 7 wherein the containment member is attached to a back portion of the garment.

9. A wearable device as in claim 7 wherein the at least one containment member further comprises a hook and loop fastener.

10. A wearable device as in claim 6 wherein the flexible securing member may stop the individual before reaching a distance away from the medical device equal to the length of the tubing connecting the individual to the medical device.

11. A wearable device as in claim 10 wherein the wearable device may be reversibly placed onto and removed from the individual without interfering with a treatment performed by the medical device and without interfering with the tubing connecting the individual to the medical device.

12. A wearable device as in claim 6 wherein the first end of the flexible securing member removably detaches from the garment.

13. A wearable device as in claim 6 wherein the flexible securing member automatically extends and retracts.

14. A wearable device as in claim 6 further comprising multiple reversibly joinable fastening members to secure the garment to the individual.

15. A wearable device as in claim 6 further comprising at least one pocket.

16. A wearable device as in claim 6 further comprising at least one slit.

17. A wearable device as in claim 6 for use with a human being.

18. A wearable device as in claim 16 for use with a child.

19. A wearable device as in claim 6 for use with an animal.

20. A wearable device as in claim 6 for use with an intravenous stand and an intravenous tubing.
